(19) United States
(12) Patent Application Publication
(10) Pub. No.: US 2014/0286887 A1
(43) Pub. Date: Sep. 25, 2014

(54) STABLE, LONG WEARING COSMETIC COMPOSITIONS COMPRISING FILM FORMING POLYMERS AND A MICROCRYSTALLINE WAX

(71) Applicant: L'OREAL, Paris (FR)

(72) Inventors: Ashlee SANDLER, Plainsboro, NJ (US); Angeles FONOLLA-MORENO, Westfield, NJ (US)

(73) Assignee: L'OREAL, Paris (FR)

(21) Appl. No.: 13/848,794

(22) Filed: Mar. 22, 2013

(51) Int. Cl. A61K 8/92; A61K 8/84; A61Q 5/00; A45D 40/26; A61K 8/89

(52) U.S. Cl. CPC A61K 8/92; A45D 40/26; A61K 8/89; A61Q 5/00; A61K 8/84
USPC 424/70.7; 401/118; 424/70.6; 424/70.12; 424/70.121

(57) ABSTRACT

Disclosed herein are cosmetic compositions comprising at least one hydrocarbon film-forming polymer, at least one silicone film-forming polymer, at least one microcrystalline wax, present in an amount ranging from about 4% to less than or equal to 8% by weight, relative to the total weight of the composition, and at least one volatile solvent. Also disclosed herein are methods for making up and/or enhancing the appearance of keratinous fibers comprising applying said compositions to the keratinous fibers.

### FIELD OF THE DISCLOSURE

The disclosure relates to cosmetic compositions comprising a hydrocarbon film-forming polymer, a silicone film-forming polymer, a microcrystalline wax, and a volatile solvent. Cosmetic compositions according to various embodiments of the disclosure are stable and when applied onto keratinous fibers, for example, the eyelashes, may provide one or more improved properties such as improved thickening, lengthening, and/or curling of the eyelashes, improved lash separation, improved resistance to clumping and/or smudging, and/or improved ease of removal. The disclosure further relates to methods for making up and/or enhancing the appearance of keratinous fibers comprising applying said composition to the keratinous fibers.

### BACKGROUND

Cosmetic compositions which enhance the appearance of human keratinous fibers such as eyelashes, eyebrows and hair, including false eyelashes and hair pieces are highly desirable to consumers. In particular, the compositions of the invention may be makeup compositions, makeup bases, compositions for applying on makeup, also known as topcoats, or even cosmetic treatment compositions for treating keratinous fibers. More generally, the invention relates to a mascara.

Mascara compositions are known and used in the cosmetic field to impart thickness, color, and/or length to the eyelashes. Several different mascaras have been developed in the art using various cosmetic ingredients depending on the desired cosmetic properties. For instance, it is known in the art that inclusion of one or more film formers in a mascara composition can improve various properties, such as the ability of the composition to thicken the eyelashes. It is also known, for example, that inclusion of fibers may enhance the lengthening properties of a mascara composition.

However, there still exists a need in the cosmetic art for “clean volume” mascara compositions, e.g., compositions that lengthen and separate the lashes and impart a smooth and homogeneous deposit, while also volumizing or thickening the lashes without clumping. As such, there is a continuous need to invent novel cosmetic compositions which demonstrate one or more of the above-mentioned improved properties and at the same time, are stable and/or demonstrate desirable textures.

It has now been surprisingly discovered that by incorporating (1) at least one hydrocarbon film-forming polymer, (2) at least one silicone film-forming polymer, and (3) at least one microcrystalline wax into a cosmetic composition, a stable composition and cosmetic properties such as improved thickening or volume, and a smooth and homogeneous deposit.

### SUMMARY OF THE INVENTION

The present invention relates to cosmetic compositions for making up and/coating keratinous fibers, said composition containing:
(a) at least one hydrocarbon film forming polymer,
(b) at least one silicone film forming polymer,
(c) at least one microcrystalline wax, present in an amount ranging from about 4% to less than or equal to 8% by weight, relative to the total weight of the composition; and
(d) at least one volatile solvent.

Furthermore, the present invention relates to a method of making up and/or enhancing the appearance of keratinous fibers comprising applying to the keratinous fibers, the above described composition.

In various embodiments of the disclosure, the present invention relates to cosmetic compositions for making up and/or coating the eyelashes and/or eyebrows comprising at least one at least one hydrocarbon film-forming polymer; at least one silicone film-forming polymer; at least one microcrystalline wax; and at least one volatile solvent. Preferably, the cosmetic composition is a mascara composition. Preferably, the cosmetic composition is anhydrous.

The present invention also relates to methods of treating, coating, caring for and/or making up eyelashes and/or eyebrows comprising applying the cosmetic compositions of the present invention to eyelashes and/or eyebrows in an amount sufficient to treat, care for and/or make up the eyelashes and/or eyebrows. Preferably, the cosmetic composition is a mascara composition. Preferably, the cosmetic composition is anhydrous.

The present invention also relates to methods of improving the stability of a cosmetic composition for eyelashes and/or eyebrows, comprising adding to said composition at least one at least one hydrocarbon film-forming polymer; at least one silicone film-forming polymer; at least one microcrystalline wax; and at least one volatile solvent. Preferably, the cosmetic composition is a mascara composition. Preferably, the cosmetic composition is anhydrous

Moreover, the present invention relates to methods of improving the volumizing and/or thickening, lengthening, lash separating, water resistance, resistance to clumping and/or smudging properties of a cosmetic composition onto eyelashes and/or eyebrows, comprising adding to said composition at least one at least one hydrocarbon film-forming polymer; at least one silicone film-forming polymer; at least one microcrystalline wax; and at least one volatile solvent. Preferably, the cosmetic composition is a mascara composition. Preferably, the cosmetic composition is anhydrous. In some embodiments, the cosmetic compositions of the present disclosure are water proof and/or water resistant and/or smudge-resistant.

In other embodiments, the present invention relates to methods of improving the homogeneity of deposition and ease of application of a make up composition onto keratinous fibers by providing a cosmetic composition of the present invention. Preferably, the cosmetic composition is a mascara composition. Preferably, the cosmetic composition is anhydrous.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

The term “keratinous fibers” means the eyelashes, the eyebrows, bodily hair or head hair.

The term “mascara” means a composition intended to be applied to keratinous fibers, especially to the eyelashes: it may be an eyelash makeup composition, an eyelash makeup base (also known as a base coat), a composition to be applied onto a mascara (also known as a top coat), or a cosmetic eyelash treatment composition. The mascara is more particularly intended for human eyelashes, but also false eyelashes.

The most conventional mascaras usually have a pasty texture and are conditioned in a container comprising a reservoir equipped with a drainer and an applicator, especially in the form of a brush or a comb, and which are applied by taking up product in the reservoir using the applicator, passing the applicator through the drainer in order to remove the excess product, and then placing the applicator impregnated with mascara in contact with the eyelashes.

“Film former” or “film forming polymer” as used herein means a polymer or resin that leaves a film on the substrate to which it is applied, for example, after a solvent accompanying the film forming polymer has evaporated, absorbed into and/or dissipated on the substrate.

“Anhydrous” means the compositions contain less than 2 percent by weight of water, or less than 1 percent by weight of water or even less than 0.5 percent by weight of water, and in particular, are exempt of water.

Where appropriate, equally small quantities of water may in particular be contributed by ingredients of the composition that may contain residual quantities of water.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

“Waterproof” or “water resistant” as used herein refers to the ability to repel water and permanence with respect to water. Waterproof or water resistance properties may be evaluated by any method known in the art for evaluating such properties. For example, when a composition is a mascara composition, it may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes. In a preferred embodiment of the present invention, little or no composition is transferred from the wearer.

“Smudge Resistant” as used herein refers to the ability to repel oil, such as a hydrocarbon oil and permanence with respect to the oil. Smudge Resistant properties may be evaluated by any method known in the art for evaluating such properties. For example, when a composition is a mascara composition, it may be applied to false eyelashes, which may then be placed in oil for a certain amount of time, such as, for example, 20 minutes. Upon expiration of the pre-ascertained amount of time, the false eyelashes may be removed from the oil and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in oil for a certain amount of time. The amount of composition remaining on the skin after the pre-ascertained amount of time may then be evaluated and compared. For example, a composition may be smudge resistant if a majority of the product is left on the wearer, e.g., eyelashes, skin, etc. In a preferred embodiment of the present invention, little or no composition is transferred from the wearer.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100 degrees C.

“Non-volatile”, as used herein, means having a flash point of greater than about 100 degree C.

The compositions and methods of the present invention can comprise, consist of, or consist essentially of the essential elements and limitations of the invention described herein, as well as any additional or optional ingredients, components, or limitations described herein or otherwise useful.

It was unexpectedly and surprisingly discovered that the cosmetic composition of the present invention, when applied as a mascara composition onto keratinous fibers such as eyelashes, formed a film or coating on said fibers that provided greater volume and length, as well as increased wear and color intensity, particularly in embodiments where the mascara is waterproof.

It was unexpectedly and surprisingly discovered that the combination of a hydrocarbon film forming polymer, a silicone film forming polymer, a microcrystalline wax, and a volatile solvent produced a composition which, when applied onto keratinous fibers, had improved thickening or volumizing, lengthening, lash separating, water resistance, resistance to clumping and/or smudging properties. Said composition was also surprisingly found to be easy to apply and deposited a homogenous coating on keratinous fibers such as the eyelashes. In preferred embodiments, said microcrystalline wax has particular physical properties related to penetration hardness and melting point and is employed in the cosmetic compositions of the present invention at certain percentages by weight.

It was also unexpectedly and surprisingly discovered that when a microcrystalline wax, having particular physical properties related to penetration hardness and melting point, was employed in the cosmetic compositions of the present invention at certain percentages by weight, said compositions were stable. The meaning of the term “stable” or “stability” as used herein means that zero or very minimal separation or breakage of a portion of the composition from the rest of the composition can be visually observed. It can also mean that the cosmetic composition of the present invention did not exhibit a phase separation and/or syneresis, that is, there is no visible crystallization or separation of the composition into two or more phases or layers. It can also mean that the cosmetic composition of the present invention did not exhibit a visible crystallization of any ingredient, such as the at least one microcrystalline wax, in the composition.

### Hydrocarbon Film Forming Polymer

The composition of the invention includes at least one hydrocarbon film forming polymer. Preferably, said film forming polymer is soluble in the at least one volatile solvent of the present invention.

Advantageously, the at least one hydrocarbon film forming polymer of the invention is a hydrocarbon “tackifier” resin. In particular, such resins are described in the Handbook of Pressure Sensitive Adhesive, edited by Donatas Satas, 3rd ed., 1989, pp. 609-619.

Said hydrocarbon film forming polymer is preferably an olefin polymer or copolymer or an aromatic hydrocarbon monomer polymer or copolymer, preferably an indene hydrocarbon resin, e.g. as described below. Said hydrocarbon film forming polymer may be hydrogenated, partially hydrogenated, or non-hydrogenated.

Preferably, said hydrocarbon film forming polymer presents a softening point that is less than 120 degrees centigrade, preferably less than 110 degrees centigrade.

In the context of the present invention, the softening point is measured using the ring-and-ball method in accordance with the ASTM D36 Standard. To do this, use is made of an automatic NBA 440 tester available from Normalab. The fluid used for the measurement is glycerin.

The hydrocarbon resins of the invention are selected from polymers that may, depending on the type of monomer that they contain, be classified as:

(a) indene hydrocarbon resins such as the resins derived from the polymerization of indene monomer in the greater proportion, and of a monomer selected from styrene, methylindene, methylstyrene, and mixtures thereof in the lesser proportion. These resins may possibly be hydrogenated. They may present a molecular weight lying in the range 290 grams per mole (g/mol) to 1150 g/mol;

Examples of indene resins that may be mentioned are those sold under the reference NORSOLENE® S95, NORSOLENE® S105, NORSOLENE® S115 by the Supplier Cray Valley, or hydrogenated styrene/methylstyrene/indene copolymers sold under the name “REGALITE™” by the supplier Eastman Chemical, in particular REGALITE™ C6100, REGALITE™ C6100L, REGALITE™ R1090, REGALITE™ R1100, REGALITE™ R7100, REGALITE™ R9100, REGALITE™ S1100, REGALITE™ S5100, or under the name ARKON® P-90, ARKON® P-100, ARKON® P-115, ARKON® M-90, ARKON® M-100, ARKON® M-115 by the supplier Arakawa.

(b) aliphatic pentadiene resins such as that derived from the polymerization mainly of 1,3-pentanediene (trans or cis piperylene) and of a minor monomer selected from isoprene, butene, 2-methyl-2-butene, pentene, 1,4-pentadiene and mixtures thereof. These resins may present a molecular weight lying in the range 1000 g/mol to 2500 g/mol.

By way of example, such 1,3-pentadiene resins are sold under the references PICCOTAC™ 95 by the supplier Eastman Chemical, ESCOREZ® 1102, ESCOREZ® 1304, ESCOREZ® 1310LC, ESCOREZ® 1315 by the supplier Exxon Chemicals, WINGTACK® 95 by the supplier Cray Valley;

(c) mixed pentadiene and indene resins that are derived from the polymerization of a mixture of pentadiene and indene monomers such as those described above, such as for example the resins sold under the reference ESCOREZ® 2101, ESCOREZ® 2105, ESCOREZ® 2173, ESCOREZ® 2184, ESCOREZ® 2203LC, ESCOREZ® 2394, ESCOREZ® 2510 by the supplier Exxon Chemicals, NORSOLENE® A 100 by the supplier Cray Valley, the resins sold under the reference WINGTACK® 86, WINGTACK® EXTRA and WINGTACK® PLUS by the supplier Cray Valley;

(d) polycyclopentadienes such as those having the reference KOBOGUARD 5400 sold by the supplier KOBO;

(e) diene resins from cyclopentadiene dimers, such as those derived from the polymerization of a first monomer selected from indene and styrene, and a second monomer selected from dimers of cyclopentadiene such as dicyclopentadiene, methyldicyclopentadiene, other dimers of pentadiene, and mixtures thereof. These resins generally present a molecular weight lying in the range 500 g/mol to 800 g/mol, such as for example those sold under the reference ESCOREZ® 5380, ESCOREZ® 5300, ESCOREZ® 5400, ESCOREZ® 5415, ESCOREZ® 5490, ESCOREZ® 5600, ESCOREZ® 5615, ESCOREZ® 5690, by the supplier Exxon Mobil Chem., and the resins SUKOREZ® SU-90, SUKOREZ® SU-100, SUKOREZ® SU-110, SUKOREZ® SU-100S, SUKOREZ® SU-200, SUKOREZ® SU-210, SUKOREZ® SU-490, SUKOREZ® SU-400, by the supplier Kolon;

(f) diene resins from isoprene dimers such as the terpene resins derived from the polymerization of at least one monomer selected from a-pinene, beta-pinene, limonene, styrene, and mixtures thereof. These resins may present a molecular weight lying in the range 300 g/mol to 2000 g/mol. By way of example, such resins are sold under the name PICCOLYTE® A115 by the supplier Hercules, ZONAREZ® 7100 or ZONATAC® 105 LITE by the supplier ARIZONA Chem.

Mention may also be made of hydrogenated resins derived mainly from the polymerization of pentadiene such as those sold under the name EASTOTAC® H-100E, EASTOTAC® H-115E, EASTOTAC® C-100L, EASTOTAC® C-115L, EASTOTAC® H-100L, EASTOTAC® H-115L, EASTOTAC® C-100R, EASTOTAC® C-115R, EASTOTAC® H-100R, EASTOTAC® H-115R, EASTOTAC® C-100W, EASTOTAC® C-115W, EASTOTAC® H-100W, EASTOTAC® H-115W, by the supplier Eastman Chemical Co.

In particular exemplary embodiments, the at least one hydrocarbon film forming polymer of the present invention is selected from indene hydrocarbon resins sold under the name NORSOLENE® S95, NORSOLENE® 5105, NORSOLENE® S115 by the supplier Cray Valley, or under the name “REGALITE™” by the supplier Eastman Chemical, in particular REGALITE™ C6100, REGALITE™ C6100L, REGALITE™ R1090, REGALITE™ R1100, REGALITE™ R7100, REGALITE™ R9100, REGALITE™ S1100, REGALITE™ S5100, or under the name ARKON® P-90, ARKON® P-100, ARKON® P-115, ARKON® M-90, ARKON® M-100, ARKON® M-115 by the supplier Arakawa.

The at least one hydrocarbon film forming polymer may be employed in the cosmetic composition of the present invention in an amount ranging from about 0.5 to about 20% by weight, or from about 1 to about 12% by weight, or from about 1 to about 10% by weight, or from about 2 to about 7.5% by weight, or from about 3 to about 5% by weight, relative to the total weight of the composition, including all ranges and subranges therebetween.

### Silicone Film Forming Polymers

The composition of the invention includes at least one silicone film forming polymer. Preferably, the at least one silicone film forming polymer is chosen from silicone resins which are cross-linked polyorganosiloxane polymers. The nomenclature of silicone resins is known under the name “MDTQ”, the resin being described as a function of the various monomeric siloxane units it includes, each of the letters “MDTQ” characterizing one type of unit.

Examples of commercially available polymethylsilsesquioxane resins that may be mentioned are those that are sold by the supplier Wacker under the reference Resin MK such as Belsil® PMS MK, and by the supplier SHIN-ETSU under the references KR-220L.

Examples of commercially available polypropylsilsesquioxane resins that may be mentioned are those that are sold under the reference DC®670 by the supplier Dow Corning.

Siloxysilicate resins that may be mentioned are trimethylsiloxysilicate resins (TMS®) such as those sold under the reference SR1000 by the supplier Momentive Performance Materials or under the reference TMS® 803 by the supplier Wacker. It is also possible to mention trimethylsiloxysilicate resins sold in a solvent such as cyclomethicone, sold under the name “KF-7312J” by the supplier Shin-Etsu, or “DC® 749”, “DC® 593” by the supplier Dow Corning.

It is also possible to mention silicone resin copolymers such as the pressure-sensitive adhesive copolymers sold in particular by the supplier Dow Corning under the reference BIO-PSA and described in the document U.S. Pat. No. 5,162,410, or silicone copolymers derived from the reaction of a silicone resin such as those described above and of a diorganosiloxane such as that described in the document WO 2004/073626.

In particular exemplary embodiments, the at least one silicone film forming polymer of the present invention is selected from siloxysilicate resins. In certain preferred embodiments, the at least one silicone film forming polymer of the present invention is a trimethylsiloxysilicate resin.

The at least one silicone film forming polymer may be employed in the cosmetic composition of the present invention in an amount ranging from about 0.1 to about 12% by weight, or from about 0.5 to about 10% by weight, or from about 0.5 to about 5% by weight, or from about 0.75 to about 3% by weight, or from about 1 to about 2.5% by weight, relative to the total weight of the composition, including all ranges and subranges therebetween.

### Microcrystalline Wax

The composition of the invention includes at least one microcrystalline wax.

As used herein, the term “wax,” is understood to mean a lipophilic fatty compound that is solid at room temperature (25 degrees C.) and atmospheric pressure (760 mmHg, i.e. 10 5 Pa), with a reversible solid/liquid change of state, having a melting point of greater than 30 degrees C., for example, greater than 55 degrees C., which may be up to 200 degrees C., for instance up to 120 degrees C.

Waxes or wax particles may be characterized by their melting point and hardness or penetration hardness properties.

For the purposes of the present disclosure, the melting point is the temperature of the most endothermic peak observed by thermal analysis (DSC) as described in ISO standard 11357-3; 1999. The melting point of a wax may be measured using a differential scanning calorimeter (DSC), for example the calorimeter sold under the name “MDSC 2920” by the company TA Instruments. For waxes that are derived from petroleum, such as microcrystalline wax, the melting point may be measured according to the drop ASTM method, D-127.

In general, the hardness of a wax may be determined by measuring the compression force, which is measured at 20 degrees C. using the texturometer sold under the name TA-XT2i® by the company Rheo, equipped with a cylindrical stainless-steel spindle 2 mm in diameter, by measuring the change in force (compression force or stretching force) (F) as a function of time, during the following operation: The spindle is displaced at a speed of 0.1 mm/s and then penetrates into the wax to a penetration depth of 0.3 mm. When the spindle has penetrated the wax to a depth of 0.3 mm, the spindle is held stationary for 1 second (corresponding to the relaxation time) and is then withdrawn at a speed of 0.1 mm/s. During the relaxation time, the force (compression force) decreases greatly until it becomes zero, and then, during the withdrawal of the spindle, the force (stretching force) becomes negative before rising again to the value 0. The hardness corresponds to the maximum compression force measured between the surface of the spindle and the wax at the moment that they come into contact. The value of this force is expressed in MPa.

To perform the hardness measurement, the wax is melted at a temperature equal to the melting point of the wax+20 degrees C. The molten wax is poured into a container 30 mm in diameter and 20 mm deep. The wax is recrystallized at room temperature (25 degrees C.) for 24 hours and is then stored for at least 1 hour at 20 degrees C. before performing the hardness measurement.

For waxes that are derived from petroleum, such as microcrystalline wax, the hardness may be measured according to the ASTM method for needle penetration of petroleum waxes, D-1321 @100/77/5. This method employs a penetrometer which measures the consistency or hardness of a semiliquid to semisolid material based on the penetration force and depth at which a cone or needle goes into the material. A penetration value of 80 corresponds to a penetration depth of 8.0 mm.

In certain embodiments, the at least one microcrystalline wax that may be used in the composition of the present invention may be chosen from the microcrystalline waxes sold by the company Strahl and Pitsch under a reference S&P number, such as SP96, SP18, SP19, SP26, SP60W, SP60, SP16, SP617, SP89 and SP624 (referred to herein as “SP microcrystalline waxes”).

The melting points of the SP microcrystalline waxes are measured according to the drop ASTM method, D-127.

The penetration hardness values of the SP microcrystalline waxes are determined according to the ASTM method, D-1321 @100/77/5.

Thus, the at least one microcrystalline wax present in the cosmetic composition as disclosed herein have a melting point of greater than or equal to 60° C. to less than 100° C., such as from between about 63° C. to about 94° C., or from between about 63° C. to about 68° C. (SP96), or from between about 82° C. to about 88° C. (SP16), or from between about 79° C. to about 85° C. (SP60), or from between about 77° C. to about 82° C. (SP19), or from between about 74° C. to about 79° C. (SP18).

The at least one microcrystalline wax of the cosmetic composition as disclosed herein can also have, for example, a penetration hardness value ranging from about 4 to about 80, for example, from about 13 to about 19 (SP16), or from about 16 to about 22 (SP60), or from about 27 to about 33 (SP19), or from about 60 to about 80 (SP18).

The at least one microcrystalline wax present in the composition as disclosed herein may also have a tack of greater than or equal to 0.7 N·s, for instance, ranging from 0.7 N·s to 30 N·s, such as greater than or equal to 0.8 N·s, for example, ranging from 0.8 N·s to 10 N·s, such as ranging from 0.8 N·s to 5 N·s.

The tack of the at least one microcrystalline wax may be measured at 20 degrees C. using the texturometer sold under the name TA-XT2i® by the company Rheo, equipped with an acrylic polymer spindle in the form of a cone forming an angle of 45 degrees, by measuring the change in force (compression force or stretching force) (F) as a function of time, during the following operation: The spindle is displaced at a speed of 0.5 mm/s and then penetrates into the wax to a penetration depth of 2 mm. When the spindle has penetrated the wax to a depth of 2 mm, the spindle is held stationary for 1 second (corresponding to the relaxation time) and is then withdrawn at a speed of 0.5 mm/s. During the relaxation time, the force (compression force) decreases greatly until it becomes zero, and then, during the withdrawal of the spindle, the force (stretching force) becomes negative before rising again to the value 0. The tack corresponds to the integral of the curve of the force as a function of time for the portion of the curve corresponding to the negative force values (stretching force). The tack value is expressed in N·s.

To perform the tack measurement of the wax, the wax is melted at a temperature equal to the melting point of the wax+10 degrees C. The molten wax is poured into a container 25 mm in diameter and 20 mm deep. The wax is recrystallized at room temperature (25 degrees C.) for 24 hours such that the surface of the wax is flat and smooth, and the wax is then kept for at least 1 hour at 20 degrees C. before performing the tack measurement.

A preferred microcrystalline wax that may be used in the composition of the present invention is the microcrystalline wax sold under the reference SP18 by the company Strahl and Pitsch.

The at least one microcrystalline wax may be employed in the cosmetic composition of the present invention in an amount ranging from about 4% to less than or equal to 8% by weight, preferably, from about 5% to less than or equal to 7.5% by weight, or more preferably, from about 6% to less than or equal to 7.5% by weight, or even more preferably, from about 6.5% to less than or equal to 7.5% by weight, relative to the total weight of the composition, including all ranges and subranges therebetween.

The at least one microcrystalline wax may also be employed in the cosmetic composition of the present invention in any of the following amounts: 7%, 7.1%, 7.2%, 7.3%, 7.4%, 7.5% by weight, relative to the total weight of the composition.

### Volatile Solvent

The composition of the invention includes at least one volatile solvent.

Preferably, the at least one hydrocarbon film forming polymer and/or the at least one silicone film forming polymer and/or the at least one microcrystalline wax of the present invention are soluble or dispersible in the at least one volatile solvent.

Representative volatile solvents of the present invention include non-polar volatile hydrocarbon-based oils (which as used herein, refers to oil containing only hydrogen and carbon atoms), silicone oils (optionally comprising alkyl or alkoxy groups that are pendant or at the end of a silicone chain), polar volatile solvents, volatile esters, volatile ethers, and fluoro oils.

Suitable hydrocarbon-based oils include isoparaffins, i.e., branched alkanes containing 8-16 carbon atoms, such as isododecane (also known as 2,2,4,4,6-pentamethylheptane), isodecane, and for example, the oils sold under the trade names of Isopar or Permethyl. Preferably, the volatile non-silicone oils have a flash point of at least 40 degrees centigrade.

Non-limiting examples of volatile non-silicone volatile oils are given below:

| | Flash Point |
|---|---|
| Compound | (° C.) |
| Isododecane | 43 |
| propylene glycol n-butyl ether | 60 |
| ethyl 3-ethoxypropionate | 58 |
| propylene glycol methylether acetate | 46 |
| Isopar L (isoparaffin C11-C13) | 62 |
| and Isopar H (isoparaffin C11-C12) | 56 |

The volatility of the solvents/oils can be determined using the evaporation speed as set forth in U.S. Pat. No. 6,338,839, the contents of which are incorporated by reference herein.

Suitable silicone oils may include linear or cyclic silicones containing from 2 to 7 silicon atoms, these silicones optionally comprising alkyl or alkoxy groups containing from 1 to 10 carbon atoms. Mention may thus be made especially of octamethylcyclotetrasiloxane, decamethylcyclopentasiloxane, hexadecamethylcyclohexasiloxane, heptamethylhexyltrisiloxane hexamethyldisiloxane, decamethyltetrasiloxane, dodecamethylpentasiloxane, heptamethyloctyltrisiloxane, decamethyltetrasiloxane, and their mixtures. Other volatile oils which may be used include KF 96A of 6 cSt viscosity, a commercial product from Shin Etsu having a flash point of 94 degrees centigrade and polydimethylsiloxanes (PDMS™) such as those available from Dow Corning under the tradename DC® 200. Preferably, the volatile silicone oils have a flash point of at least 40 degrees centigrade, and mixtures thereof. Mixtures of these solvents may be used.

Polar volatile solvents may also be used, examples of which include C2 to C5 alcohols, such as ethanol, ethyl 3-ethoxypropionate and isohexyl neopentanoate.

A preferred volatile solvent that may be used in the cosmetic compositions of the present invention is isododecane.

The at least one volatile solvent is present in the cosmetic composition of the present invention in an amount generally ranging up to about 95% by weight, and in some embodiments, from about 10% to about 95% by weight, and in other embodiments, from about 20% to about 90% by weight, or from about 30% to about 80% by weight, or from about 40% to about 70% by weight, relative to the total weight of the cosmetic composition.

### Non-Volatile Solvents

The compositions of the present invention may also comprise non-volatile solvents such as non-polar non-volatile solvents.

Exemplary non-polar non-volatile solvents include polyalphaolefins, which include ethylene derivatives oligomerized into even-numbered carbon polyalphaolefins e.g., C6-C14 olefins such as polydecene and polymers of C6, C8, C12 and C14 olefins. The polyolefins may have a molecular weight (MW) generally ranging from about 280 to about 11,500, and a viscosity (CPs at 20 degrees C.) generally ranging from about 7 to about 32,500. They may also be hydrogenated. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively) PureSyn™ 100 (MW about 2939, viscosity about 3900, INCI name: hydrogenated C6-14 olefin polymers) and PureSyn™ 1000 (MW about 11,500, viscosity about 32,400, INCI name: polydecene) may also be useful. The PureSyn™ products are available from Exxon Chemicals.

The non-volatile solvent is present in the cosmetic composition of the present invention in an amount generally ranging from about 0.1% to about 70% by weight, and in some embodiments, about 0.5% to about 40% by weight, and in other embodiments, about 1% to about 10% by weight, relative to the total weight of the cosmetic composition.

The inventive compositions may contain any other cosmetically or dermatologically acceptable and, in general, physiologically acceptable oil, such as carbon-based, hydrocarbon-based, fluoro and/or silicone oils, of mineral, animal, plant or synthetic origin, alone or as a mixture. These ingredients, along with the non-polar solvents, could comprise a liquid fatty phase or oil carrier of the cosmetic composition of the present invention.

According to other embodiments of the present invention, the oil carrier comprises at least one non-volatile oil. Examples of non-volatile oils that may be used in the present invention include, but are not limited to, polar oils such as:

hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;

synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C12 to C15 alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;

synthetic ethers containing from 10 to 40 carbon atoms;

C8 to C26 fatty alcohols, for instance oleyl alcohol, cetyl alcohol, stearyl alcohol, and cetearyl alcohol; and

mixtures thereof.

Further, examples of non-volatile oils that may be used in the present invention include, but are not limited to, non-polar oils such as branched and unbranched hydrocarbons and hydrocarbon waxes including polyolefins, in particular, Vaseline (petrolatum), paraffin oil, squalane, squalene, hydrogenated polyisobutene, hydrogenated polydecene, polybutene, mineral oil, pentahydrosqualene, and mixtures thereof.

### Desired Agents

According to preferred embodiments of the present invention, the compositions can further comprise a desired agent. The desired agent can be, for example, a colorant (pigment, dye, etc.), a fiber, an additional wax other than the at least one microcrystalline wax of the present invention and as described above, a cosmetically active agent, or a film forming agent known in the art other than the at least one hydrocarbon film forming polymer and the at least one silicone film forming polymer of the present invention and as described above. For example, a cosmetic makeup composition of the present invention and further comprising a colorant can provide a desired color to a substrate (eyelashed, eyebrows, etc.) during use. Similarly, the cosmetic composition of the present invention further comprising a cosmetically active agent can provide such active agent to the consumer upon use.

Acceptable colorants include pigments, dyes, such as liposoluble dyes, nacreous pigments, and pearling agents.

Representative liposoluble dyes which may be used according to the present invention include Sudan Red, DC® Red 17, DC® Green 6, beta-carotene, soybean oil, Sudan Brown, DC® Yellow 11, DC® Violet 2, DC® Orange 5, annatto, and quinoline yellow.

Representative nacreous pigments include white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

Representative pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D and C type, and lakes based on cochineal carmine, barium,

Suitable fibers include, but are not limited to, fibers which enable improvement of the thickening/volumizing and/or lengthening effect. As used herein, the term “fiber” is understood to mean an object of length L and diameter D such that L is greater than D, for instance, L may be very much greater than D, wherein D is the diameter of the circle in which the cross section of the fiber is inscribed. For example, the ratio of L to D (or shape factor) can range from 3.5 to 2,500, for instance, from 5 to 500, such as from 5 to 150.

The fibers that can be used in the composition of the present disclosure may be mineral or organic fibers of synthetic or natural origin. They may be short or long, individual or organized, for example braided, and hollow or solid. They may have any shape, and may have, for example, a circular or polygonal (square, hexagonal or octagonal) cross section, depending on the intended specific application. In one embodiment of the present disclosure, the fibers' ends are blunt and/or polished to prevent injury.

For example, the fibers can have a length ranging from 1 mu m to 10 mm, for instance from 0.1 mm to 5 mm, such as from 0.3 mm to 3 mm. Their cross section may be within a circle of diameter ranging from 2 nm to 500 mu m, for instance ranging from 100 nm to 100 mu m, such as from 1 mu m to 50 mu m. The weight or yarn count of the fibers is often given in denier or decitex, and represents the weight in grams per 9 km of yarn. For example, the fibers according to the present disclosure may have a yarn count ranging from 0.01 denier to 10 denier, for instance from 0.1 denier to 2 denier, such as from 0.3 denier to 0.7 denier.

The fibers that can be used include those used in the manufacture of textiles, such as silk fiber, cotton fiber, wool fiber, flax fiber, cellulose fiber extracted for instance, from wood, from legumes or from algae, rayon fiber, polyamide (Nylon®) fiber, viscose fiber, acetate fiber, such as rayon acetate fiber, acrylic polymer fiber, for instance: polymethyl methacrylate fiber or poly(2-hydroxyethyl methacrylate) fiber, polyolefin fiber such as polyethylene or polypropylene fiber, glass fiber, silica fiber, carbon fiber, for instance in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers such as those mentioned above, for instance polyamide/polyester fibers.

In one embodiment of the present disclosure, the fibers are polyamide (Nylon®) fibers.

The fibers used in surgery can also be used, for instance the resorbable synthetic fibers prepared from glycolic acid and caprolactone (Monocryl from Johnson Johnson); resorbable synthetic fibers of the type which is a copolymer of lactic acid and of glycolic acid (Vicryl from Johnson Johnson); polyterephthalic ester fibers (Ethibond from Johnson Johnson) and stainless steel threads (Acier from Johnson Johnson).

Moreover, the fibers may or may not be surface-treated, and may or may not be coated with a protective coat. Among coated fibers that may be used as disclosed herein, non-limiting mention may be made of polyamide fibers coated with copper sulphide to give an anti-static effect (for example R-STAT from Rhodia) or another polymer enabling a particular organization of the fibers (specific surface treatment). Non-limiting mention may also be made of fibers coated with mineral or organic pigments, such as the pigments described herein below.

For example, in another embodiment of the present disclosure, fibers of synthetic origin, for instance organic fibers, such as those used in surgery, are used.

For further example, in still another embodiment of the present disclosure, the fibers may be chosen from polyamide fibers, cellulose fibers and polyethylene fibers. Their length (L) may range from 0.1 mm to 5 mm such as from 0.25 mm to 1.6 mm, and their mean diameter may range from 1 mu m to 50 mu m. For instance, the polyamide fibers sold by Etablissements P. Bonte under the name “Polyamide 0.9 Dtex 3 mm,” having a mean diameter of 6 mu m, a yarn count of about 0.9 dtex and a length ranging from 0.3 mm to 5 mm, or the polyamide fibers sold under the name Fiberlon 931-D1-S by the company LCW, having a yarn count of about 0.9 dtex and a length of about 0.3 mm, may be used. Cellulose (or rayon) fibers with a mean diameter of 50 mu m and a length ranging from 0.5 mm to 6 mm may also be used, for instance those sold under the name “Natural rayon flock fiber RC1BE-N003-M04” by the company Claremont Flock. Polyethylene fibers, for instance those sold under the name “Shurt Stuff 13 099 F” by the company Mini Fibers, may also be used.

Elastomeric fibers may also be used, i.e. fibers which, when subjected to a stretching stress (for example of 30 percent relative to their initial length), return to a length substantially identical to their initial length when the stress is removed. Among the elastomeric fibers that may be used, non-limiting mention may be made of polyurethane fibers such as elastane (or Spandex), fibers comprising at least 85 percent by weight of segmented polyurethane, such as Lycra sold by Dupont de Nemours, elastodiene, or alternatively rubbery fibers obtained from natural rubber. These elastomeric fibers may or may not be vulcanized.

The composition according to the present disclosure may also comprise “rigid” fibers, as opposed to the fibers mentioned above, which are not rigid fibers.

The rigid fibers, which are initially substantially straight, when placed in a dispersing medium, do not undergo a substantial change in shape, which is reflected by the angular condition defined below, reflecting a shape that may be described as still substantially straight and linear. This angle condition reflects the stiffness of the fibers, which it is difficult to express by another parameter for objects that are as small as the rigid fibers.

The stiffness of the fibers is reflected by the following angular condition: for example, at least 50 percent, for instance at least 75 percent, such as at least 90 percent, in numerical terms, of the rigid fibers are such that the angle formed between the tangent to the longitudinal central axis of the fiber and the straight line connecting the said end to the point on the longitudinal central axis of the fiber corresponding to half the length of the fiber is less than 15 degrees, and the angle formed between the tangent to the longitudinal central axis of the fiber at a point half way along the fiber and the straight line connecting one of the ends to the point on the longitudinal central axis of the fiber corresponding to half the length of the fiber, is less than or equal to 15 degrees for the same fiber length ranging from 0.8 mm to 5 mm, for instance ranging from 1 mm to 4 mm, and from 1 mm to 3 mm, such as 2 mm.

For example, the angle mentioned above may be measured at the two ends of the fiber and at a point half way along the fiber; in other words, three measurements can be taken in this case and the average of the measured angles is less than or equal to 15 degrees.

The tangent, at any point on the fiber, for instance forms an angle of less than 15 degrees.

In the present disclosure, the angle formed by the tangent at a point on the fiber is the angle formed between the tangent to the longitudinal central axis of the fiber at the said point on the fiber and the straight line connecting the end of the fiber that is closest to the said point to the point on the longitudinal central axis of the fiber corresponding to half the length of the fiber.

For example, the rigid fibers that may be used in the composition as disclosed herein can have the same or substantially the same fiber length.

For instance, when a medium where rigid fibers are dispersed in an amount of 1 percent by weight, is observed by microscope, with an objective lens allowing a magnification of 2.5 and with full-field vision, a numerical majority of the rigid fibers, i.e. at least 50 percent numerically of the rigid fibers, for instance, at least 75 percent numerically of the rigid fibers such as at least 90 percent numerically of the rigid fibers, must satisfy the angular condition defined above. The measurement leading to the angle value is performed for the same length of fibers, this length ranging from 0.8 mm to 5 mm, for instance from 1 to 4 mm, and from 1 to 3 mm, such as 2 mm.

The medium on which the observation is performed is a dispersing medium that ensures good dispersion of the rigid fibers, for example water or an aqueous gel of clay or of associative polyurethane. A direct observation of the composition comprising the rigid fibers may even be performed. A sample of the prepared composition or dispersion is placed between a slide and cover slip for observation by microscope with an objective lens allowing a magnification of 2.5 and with full-field vision. Full-field vision allows the fibers to be viewed in their entirety.

The rigid fibers can be chosen from fibers of a synthetic polymer chosen from polyesters, polyurethanes, acrylic polymers, polyolefins, polyamides, for instance nonaromatic polyamides, and aromatic polyimideamides.

Non-limiting examples of rigid fibers that may be mentioned include:

polyester fibers, such as those obtained by chopping yarns sold under the names Fiber 255-100-R11-242T Taille 3 mm (eight-lobed cross section), Fiber 265-34-R11-56T Taille 3 mm (round cross section) and Fiber Coolmax 50-34-591 Taille 3 mm (four-lobed cross section) by the company Dupont de Nemours;

polyamide fibers, such as those sold under the names Trilobal Nylon® 0.120-1.8 DPF; Trilobal Nylon® 0.120-18 DPF; Nylon® 0.120-6 DPF by the company Cellusuede products; or obtained by chopping yarns sold under the name Fiber Nomex® Brand 430 Taille 3 mm by the company Dupont de Nemours;

polyimideamide fibers, such as those sold under the names “Kermel®” and “Kermel® Tech” by the company RHODIA;

poly(p-phenyleneterephthalamide) (or Aramide®) sold, for instance, under the name Kevlar® by the company Dupont de Nemours;

fibers with a multilayer structure comprising alternating layers of polymers chosen from polyesters, acrylic polymers and polyamides. Such fibers are sold under the names “Morphotex®” and “Teijin Tetron Morphotex®” by the company Teijin.

In one embodiment of the present disclosure, the rigid fibers are chosen from aromatic polyimideamide fibers.

In one aspect of the present disclosure, the aromatic polyimideamide fibers are polyimideamide fibers comprising repeating units of formula:

obtained by polycondensation of tolylene diisocyanate and trimellitic anhydride.

The fibers are present in the composition according to the present disclosure in an amount ranging from 0.05 percent to 10 percent by weight, for instance from 0.1 percent to 5 percent by weight, such as from 0.3 percent to 3 percent by weight, relative to the total weight of the composition.

The cosmetic composition as disclosed herein may further comprise at least one additional wax, other than the at least one microcrystalline wax.

The additional waxes that may be used in the compositions according to the present disclosure are chosen from waxes that are solid and rigid at room temperature, of animal, plant, mineral or synthetic origin, and mixtures thereof.

The at least one additional wax may also have a hardness ranging from 0.05 MPa to 30 MPa, for instance ranging from 6 MPa to 15 MPa, the hardness being determined by the method mentioned for the tacky wax.

Among the additional waxes that may be used, non-limiting mention may be made of hydrocarbon-based waxes such as beeswax, lanolin wax and Chinese insect waxes; rice wax, carnauba wax, candelilla wax, ouricury wax, esparto grass wax, cork fiber wax, sugar cane wax, Japan wax and sumach wax; montan wax, microcrystalline waxes, paraffins and ozokerite; polyethylene waxes, the waxes obtained by Fisher-Tropsch synthesis and waxy copolymers, and also esters thereof. Non-limiting mention may also be made of the waxes obtained by catalytic hydrogenation of animal or plant oils comprising linear or branched C8-C32 fatty chains.

Among these, further non-limiting mention may be made of, for example, hydrogenated jojoba oil, isomerized jojoba oil such as the partially hydrogenated isomerized jojoba oil manufactured or sold by the company Desert Whale under the commercial reference Iso-Jojoba™-50, hydrogenated sunflower oil, hydrogenated castor oil, hydrogenated coconut oil and hydrogenated lanolin oil, bis(1,1,1-trimethylolpropane)tetrastear-ate sold under the name “Hest 2T-4S” by the company Heterene and bis(1,1,1-trimethylolpropane)tetrabehenate sold under the name Hest 2T-4B by the company Heterene.

Non-limiting mention may also be made of silicone waxes and fluoro waxes.

It is also possible to use the wax obtained by hydrogenation of olive oil esterified with stearyl alcohol, sold under the name “Phytowax® Olive 18 L 57” or the waxes obtained by hydrogenation of castor oil esterified with cetyl alcohol, sold under the name “Phytowax® ricin 16L64 and 22L73” by the company Sophim.

The at least one additional wax may also be present in the form of a wax microdispersion as described above for the tacky wax. The at least one additional wax, when present, can be present in the composition according to the present disclosure in an amount ranging from 0.1% to 5% by weight, for instance from 0.5% to 30% by weight, such as from 1% to 20& by weight, relative to the total weight of the composition.

The film forming agent that may be used in the compositions of the present disclosure other than the at least one hydrocarbon film forming polymer and the at least one silicone film forming polymer described above may be chosen from polyalkylenes, vinyl polymers, silicone polyamides, and linear film forming ehtylenic block copolymers.

Suitable examples of polyalkylenes are copolymers of C₂-C₂₀ alkenes such as polybutene, alkylcelluloses with a C₁ to C₈, linear or branched alkyl radical, saturated or unsaturated, such as ethylcellulose or propylcellulose, copolymers of vinylpyrrolidone (VP) and in particular copolymers of vinylpyrrolidone and C₂ to C₄₀ alkene, more preferably C₃ to C₂₀. Examples of VP copolymers that may be used in the invention and that may be mentioned are the copolymer of VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene and VP/acrylic acid/lauryl methacrylate.

The film forming agent may also be a vinyl polymer including at least one motif derived from carbosiloxane dendrimer.

Vinyl polymers including motifs derived from carbosiloxane dendrimer are also suitable for use in the invention.

In particular, the vinyl polymer may have a skeleton and at least one lateral chain that comprises a carbosiloxane dendrimer structure. In the context of the present invention, the term “carbosiloxane dendrimer structure” represents a molecular structure having branched groups having high molecular weights, said structure having a high regularity in the radial direction starting from the bond to the skeleton.

Vinyl polymers grafted with at least one motif derived from carbosiloxane dendrimer that are particularly suitable for the present invention are the polymers sold under the names TIB 4-100, TIB 4-101, TIB 4-120, TIB 4-130, TIB 4-200, FA 4002 ID (TIB 4-202), TIB 4-220, and FA 4001 CM (TIB 4-230) by the supplier Dow Corning.

It is also possible to use silicone polyamides of the polyorganosiloxane type.

These silicone polymers may belong to the following two families:

polyorganosiloxanes comprising at least two groups capable of establishing hydrogen interactions, these two groups being located in the polymer chain; and/or

polyorganosiloxanes comprising at least two groups capable of establishing hydrogen interactions, these two groups being located on grafts or branches.

The film-forming agent may also be a linear film-forming ethylenic block polymer that preferably comprises at least one first block and at least one second block having different glass transition temperatures (Tg), said first and second blocks being connected together via an intermediate block comprising at least one constituent monomer of the first block and at least one constituent monomer of the second block.

Advantageously, the first and second blocks of block polymer are incompatible with each other.

### Additives

The composition of the invention may further include any additive that is usually used in cosmetics.

Naturally, the person skilled in the art takes care to select any complementary cosmetic additives and/or their quantities in such a manner that the advantageous properties of the composition of the invention is spoilt little, if at all, by the envisaged addition.

Examples of particular additives that may be mentioned are antioxidants, fillers, preservatives, fragrances, neutralizers, thickeners, vitamins, coalescers, plasticizers, and mixtures thereof.

The fillers may be present in the composition at a content lying in the range 0.01% to 50% by weight relative to the total weight of the composition, preferably lying in the range 0.1% to 30% by weight, e.g. lying in the range 1% to 25% by weight, e.g. lying in the range 5% to 20% by weight.

The term “fillers” means particles of any shape, colorless or white, inorganic or synthetic, and insoluble in the composition medium regardless of the temperature at which the composition is manufactured, to the exclusion of fibers. These fillers serve in particular to modify the rheology or the texture of the composition.

The fillers may be organic or inorganic and of any shape, flakes, spherical, or oblong, whatever the crystal shape (e.g. sheets, cubic, hexagonal, orthorombic, etc).

Mention may be made of mica, kaolin, poly-beta-alanine and polyethylene powders, tetrafluoroethylene polymer powders (Teflon®), lauroyl-lysine, starch, boron nitride, hollow polymeric microspheres such as those formed from polyvinylidene chloride/acrylonitrile, such as Expancel®(Nobel Industrie), acrylic acid copolymers (Polytrap® from the supplier Dow Corning) and silicone resin microbeads (e.g. Tospearls® from the supplier Toshiba), particles of polyorganosiloxane elastomers, precipitated calcium carbonate, magnesium carbonate and bicarbonate, hydroxyapatite, hollow silica microspheres (Silica Beads® from the supplier Maprecos), glass or ceramic microcapsules, metallic salts derived from organic carboxylic acids containing 8 to 22 carbon atoms, preferably 12 to 18 carbon atoms, for example zinc, magnesium, or lithium stearate, zinc laurate, and magnesium myristate.

In particular, they may preferably be selected from talc, silica, rice starch, polyamide powders (Nylon®), and polymethylmethacrylate powders, in particular those sold under the name COVABEAD® LH85 by the supplier LCW.

In one embodiment of the present invention is a composition which contains so little TEA-stearate that the presence of TEA-stearate does not affect the cosmetic properties of the composition. Preferably, the compositions are substantially free of TEA-stearate (i.e., contain less than about 0.5 percent TEA-stearate), essentially free of TEA-stearate (i.e., contain less than about 0.25 percent TEA-stearate) or free of TEA-stearate (i.e., contain no TEA-stearate).

According to other preferred embodiments, methods of treating, coating and/or enhancing the appearance of keratinous fibers, such as the eyelashes and/or eyebrows, by applying compositions of the present invention to keratinous fibers in an amount sufficient to treat, care for and/or enhance the appearance of the keratinous fibers are provided. In accordance with these preceding preferred embodiments, the compositions of the present invention are applied topically to the desired area in an amount sufficient to treat, care for and/or enhance the appearance of the keratinous fibers. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, improved feel upon application (for example, texture, reduced drag or tackiness), increased volume properties and/or increased wear properties, improved water resistance, smudge resistance and transfer resistance properties are provided.

According to another preferred embodiment of the present invention, compositions having improved stability such that there is zero or very minimal visual breakage of portions of the compositions from the bulk of the compositions are provided.

According to another preferred embodiment of the present invention, compositions having improved stability such that there is zero or very minimal visual crystallization of the at least one microcrystalline wax are provided.

According to certain embodiments of the present invention, methods of improving the volumizing and/or water resistance of a composition, comprising adding at least one hydrocarbon film forming polymer, at least one silicone film forming polymer, at least one microcrystalline wax and at least one volatile solvent to the composition are provided.

According to other embodiments, methods of applying the compositions of the present invention onto keratinous fibers such as eyebrows and/or eyelashes are provided. Compared with compositions (comparative) which contain waxes other than the at least one microcrystalline wax, which form harder films upon application, compositions containing the microcrystalline wax of the present invention (inventive) form a smooth and homogeneous film and are easier to apply onto the keratinous fibers.

The composition according to the present disclosure may be manufactured by the known processes generally used in cosmetics.

When the composition of the present invention is a mascara, the composition may be packaged in an applicator product comprising a reservoir and a removable cap for closing the reservoir, for example in a leaktight manner.

The applicator assembly may also comprise a member for applying the composition to keratinous fibers, such as the eyelashes, wherein the applicator member allows the composition to be taken up and also allows the composition taken up to be deposited on the eyelashes. This applicator member can be, for example, securely fastened to the cap for leaktight closure of the assembly.

The applicator assembly may also comprise a draining member (or drainer) for the applicator member, the draining member possibly being securely fastened to the reservoir.

The applicator member may for example, be a mascara brush that is well known to those skilled in the art. Such a brush for instance, comprises bristles arranged radially around a twisted core, such as a metal core. The brush may be of varied shape and may comprise cutout sections. Mascara brushes are described, for example, French Patent No. FR-A-2 607 373, and European Patent Nos. EP-A-611 170, EP-A-811 336, EP-A-811 337 and EP-A-842 620.

Alternatively, the applicator may comprise a comb comprising a plurality of teeth obtained by molding with a support made of thermoplastic material. The applicator may also comprise a comb combined with a brush.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The following Examples are intended to be non-restrictive and explanatory only, with the scope of the invention being defined by the claims.

The compositions below were prepared by mixing, independently, the components set forth in the following Tables.

### Example 1

### Inventive Formula a and Comparative Formulas, Mascara Compositions

TABLE 1
INVENTIVE COMPOSITION, FORMULA A

| | | Concentration |
|---|---|---|
| Phase | Ingredient - INCI US Name | % by weight |
| A | ISODODECANE | 45.74 |
| A1 | DISTEARDIMONIUM HECTORITE | 7.5 |
| A1 | BENZOIC ACID | 0.1 |
| A1 | IRON OXIDES | 6 |
| A1 | TALC | 4.5 |
| A1 | TRIMETHYLSILOXYSILICATE^{a} | 2.5 |
| B | COPERNICIA CERIFERA | 4.41 |
| | (CARNAUBA) WAX | |
| B | PARAFFIN | 7.11 |
| B | MICROCRYSTALLINE WAX^{b} | 7.2 |
| B | HYDROGENATED STYRENE/METHYL | 5 |
| | STYRENE/INDENE COPOLYMER^{c} | |
| C | HYDROGENATED | 4 |
| | POLYCYCLOPENTADIENE | |
| | (70% active in isododecane)^{d} | |
| D | PROPYLENE CARBONATE | 2.14 |
| E | COPERNICIA CERIFERA | 3 |
| | (CARNAUBA) WAX (and) | |
| | PEG-30 GLYCERYL STEARATE | |
| F | NYLON-6 (and) SILICA (and) BLACK 2 | 0.01 |
| F | RAYON | 0.56 |
| F | TDI/TRIMELLITIC | 0.23 |
| | ANHYDRIDE COPOLYMER | |

^{a}SR1000, commercially available from Momentive Performance Materials
^{b}White Microcrystalline wax, commercially available from Strahl & Pitsch
^{c}Regalite 1100, commercially available from Eastman Chemical
^{d}KOBOGUARD 5400 IDD, commercially available for KOBO

Process of Making Formula A:

Formula A was prepared using a Rayneri homogenizer. Phase A1 was added to phase A in a main kettle and the mixture was heated to 80° C. while A1 was dispersed in A for one hour. Phase B was heated in a side kettle to 80° C. in order to melt all the waxes into liquid form. The melted waxes were added to the main kettle and homogenized at a speed of 600 rpm for 15 minutes. Phase C was immediately added to the main kettle while the temperature of the batch was still at 80° C. Phase D was added to the main kettle. The homogenizer was then switched to a sweep blade and the batch was cooled to 45° C. Once a temperature reached a cooling point, phase E and F were added. Once the batch has cooled to 30° C., the batch was ready to be removed from the mixer and packaged.

TABLE 2
COMPARATIVE COMPOSITIONS

| | Concentration | | | | | | |
|---|---|---|---|---|---|---|---|
| Ingredient | % by weight | | | | | | |
| INCI US Name | B | C | D | E | F | G(*) | H |
| HYDROGENATED | — | — | — | 5 | — | 5 | 5 |
| STYRENE/METHYL | | | | | | | |
| STYRENE/INDENE | | | | | | | |
| COPOLYMER | | | | | | | |
| TRIMETHYLSILOXYSILICATE | — | — | — | 2.5 | — | 2.5 | 2.5 |
| MICROCRYSTALLINE | 8 | — | — | 8 | — | 9 | 3.6 |
| WAX | | | | | | | |
| ISODODECANE | 50.91 | 50.43 | 52.13 | 46.86 | 59.07 | 46.86 | 50.54 |
| DISTEARDIMONIUM | 6.5 | 5.8 | 5.3 | 6.5 | 6 | 6.5 | 7.5 |
| HECTORITE | | | | | | | |
| HYDROGENATED | 2.8 | — | — | 2.8 | 2.8 | 2.8 | 2.8 |
| POLYCYCLOPENTADIENE | | | | | | | |
| IRON OXIDES | 6 | 4.2 | | 6 | 6 | 6 | 6 |
| PROPYLENE | 2.14 | 1.9 | 1.9 | 2.14 | 1.98 | 2.14 | 2.14 |
| CARBONATE | | | | | | | |
| SYNTHETIC | — | — | — | — | 10 | — | — |
| BEESWAX AND | | | | | | | |
| EUPHORBIA | | | | | | | |
| CERIFERA | | | | | | | |
| (CANDELILLA) WAX | | | | | | | |
| PARAFFIN AND | 13.6 | 9.4 | 9.4 | 13.6 | 5 | 13.6 | 12.32 |
| COPERNICIA | | | | | | | |
| CERIFERA | | | | | | | |
| (CARNAUBA) WAX | | | | | | | |
| BEESWAX AND | — | 11.1 | 11.1 | — | — | — | — |
| ORYZA SATIVA | | | | | | | |
| (RICE) BRAN WAX | | | | | | | |
| POLYACRYLATE | — | — | — | — | 2.25 | — | — |
| CROSSPOLYMER-6 | | | | | | | |
| POLYVINYL | — | 7.5 | 7.5 | — | — | — | — |
| LAURATE, | | | | | | | |
| VP/EICOSENE | | | | | | | |
| COPOLYMER AND | | | | | | | |
| ALLYL | | | | | | | |
| STEARATE/VA | | | | | | | |
| COPOLYMER | | | | | | | |
| SUCROSE ACETATE | — | — | — | — | 5 | — | — |
| ISOBUTYRATE | | | | | | | |
| TALC | 3.5 | 1 | 1 | 3.5 | — | 3.5 | 4.5 |
| QUATERNIUM-90 | — | — | 0.5 | — | — | — | — |
| BENTONITE | | | | | | | |
| POLYGLYCERYL-3 | — | — | 0.5 | — | — | — | — |
| DIISOSTEARATE | | | | | | | |
| RAYON AND | | — | — | 0.8 | 0.8 | 0.8 | 0.8 |
| TDI/TRIMELLITIC | | | | | | | |
| ANHYDRIDE | | | | | | | |
| COPOLYMER | | | | | | | |
| ETHYLENEDIAMINE/ | 0.997 | 0.997 | 0.997 | — | — | — | — |
| STEARYL DIMER | | | | | | | |
| DILINOLEATE | | | | | | | |
| COPOLYMER | | | | | | | |
| OTHER | QS 100 | QS 100 | QS 100 | QS 100 | QS 100 | QS 100 | QS 100 |
| INGREDIENTS | | | | | | | |
| CHOSEN FROM: | | | | | | | |
| PRESERVATIVES, | | | | | | | |
| PEG-30 GLYCERYL | | | | | | | |
| STEARATE, DENAT. | | | | | | | |
| ALCOHOL, PLANT | | | | | | | |
| EXTRACTS, | | | | | | | |
| HYDROGENATED | | | | | | | |
| JOJOBA OIL, | | | | | | | |
| SILICA, | | | | | | | |
| ACRYLATES | | | | | | | |
| COPOLYMER. | | | | | | | |
| ISOBUTANE, | | | | | | | |
| PANTHENOL, | | | | | | | |
| ACRYLONITRILE/METHYL | | | | | | | |
| METHACRYLATE/VINYLIDENE | | | | | | | |
| CHLORIDE | | | | | | | |
| COPOLYMER, | | | | | | | |
| NYLON-6 (and) | | | | | | | |
| SILICA (and) | | | | | | | |
| BLACK 2 | | | | | | | |

(*)with 9% microcrystalline wax

The comparative mascara compositions were prepared similarly according to the procedure of making the inventive mascara compositions above.

### Example 2

### Stability test

The stability of the inventive formula was compared against the stability of comparative formulas E, F, G and H from Table 2. Each test formula was scooped out into a corresponding smaller jar. Each jar was placed into a controlled environment chamber set at 45° C. for two months. When the formulas were removed from the oven, they were checked for syneresis (or breakage) and crystallization and photographed. Then each formula was scooped out of the jar and weighed. The portions of the formula that resulted from syneresis and crystallization were re-weighed alone. The percentage of each formula that broke out of the dispersion was then calculated as a % of breakage of the formula.

TABLE 3
Stability test results

| | | Total | Weight of | |
|---|---|---|---|---|
| | | Weight | formula | |
| | % of | of | resulting from | |
| | Microcrystalline | sample | syneresis and | % of |
| | Wax in the | in | crystallization | breakage |
| Formula | formula | grams | in grams | of formula |
| E | 8 | 25.8 | 0.26 | 1.008 |
| F | 0 | 86.66 | 1.63 | 1.881 |
| G(*) | 9 | 31.58 | 0.84 | 2.660 |
| H | 3.6 | 44.55 | 0.64 | 1.437 |
| A(**) | 7.2 | 42.48 | 0 | 0% |

(*)with 9.0% microcrystalline wax
(**)Inventive formula)

Table 3 above demonstrates the % of breakage of each test formula; the lower the % value, the more stable the formula was. The results show that the inventive formula, which contained 7.2% microcrystalline wax, was the most stable formula because it demonstrated 0% breakage. Formula F, which had the highest amount of microcrystalline wax, was the least stable, followed by Formula F which did not contain microcrystalline wax.

Compared to formulas E and F, the inventive formula A did not visually exhibit a separation or breakage of a portion of the formula; that is, the inventive formula A appeared to be homogeneous or to be a single phase. On the other hand, the comparative formulas E and F visually showed breakage (syneresis) of portions of said formulas.

For formulas B, C, and D, it can be visually observed that white pieces of microcrystalline wax was crystallizing out of the mixture.

### Example 3

### Comparison Between the Inventive Formula and Comparative Mascara Formulas that Did not Contain Microcrystalline Wax

TABLE 4

| | FORMULA A | FORMULA I |
|---|---|---|
| Ingredient | Concentration | Concentration |
| INCI US Name | % by weight | % by weight |
| IRON OXIDES | 6 | 6 |
| COPERNICIA CERIFERA | 4.41 | 4.41 |
| (CARNAUBA) WAX | | |
| EUPHORBIA CERIFERA | | 7.2 |
| (CANDELILLA) WAX | | |
| PARAFFIN | 7.11 | 7.11 |
| MICROCRYSTALLINE WAX | 7.2 | |
| COPERNICIA CERIFERA | 3 | 3 |
| (CARNAUBA) WAX | | |
| (and) PEG-30 | | |
| GLYCERYL STEARATE | | |
| DISTEARDIMONIUM HECTORITE | 7.5 | 7.5 |
| TALC | 4.5 | 4.5 |
| RAYON | 0.56 | 0.56 |
| TDI/TRIMELLITIC ANHYDRIDE | 0.23 | 0.23 |
| COPOLYMER | | |
| HYDROGENATED | 4 | 4 |
| POLYCYCLOPENTADIENE | | |
| HYDROGENATED | 5 | 5 |
| STYRENE/METHYL | | |
| STYRENE/INDENE COPOLYMER | | |
| NYLON-6 (and) SILICA (and) | 0.01 | 0.01 |
| BLACK 2 | | |
| BENZOIC ACID | 0.1 | 0.1 |
| TRIMETHYLSILOXYSILICATE | 2.5 | 2.5 |
| PROPYLENE CARBONATE | 2.14 | 2.14 |
| ISODODECANE | 45.74 | 45.74 |

Each of the formulas in Table 4 was applied onto false eyelashes.

20 strokes of each formula were applied onto each set of false eyelashes (dipping into the mascara composition after the 10^{th} stroke). Lashes were allowed to dry for one minute before visual observations of the coated lashed were made.

It was visually observed that the eyelashes treated with Formula A had more volume and/or thicker as well as had more definition than the eyelashes coated with Formula I. This results means that the inventive formula had better thickening and/or volumizing properties.

TABLE 5
VISCOSITY DATA

| Formula | Viscosity |
|---|---|
| A | 54.9 ud* 10 min |
| I | 16.5 ud* 10 min |

*ud = units on the Rheomat RM180

The viscosity data in Table 5 shows that the inventive formula had a viscosity that was higher than the comparative formula; this degree of viscosity provided better application and coverage of the eyelashes.

TABLE 6
Comparative mascara formula that does not contain microcrystalline
wax, trimethylsiloxysilicate resin and hydrogenated styrene/methyl
styrene/indene copolymer

| | Concentration |
|---|---|
| | FORMULA J |
| Ingredient - INCI US Name | % by weight |
| ARGININE | 0.05 |
| SERINE | 0.05 |
| GLUTAMIC ACID | 0.05 |
| 2-OLEAMIDO-1,3-OCTADECANEDIOL | 0.05 |
| SOLUBLE COLLAGEN | 0.1 |
| IRON OXIDES | 7.14 |
| COPERNICIA CERIFERA (CARNAUBA) WAX | 4.7 |
| BEESWAX | 8.3 |
| PARAFFIN | 2.8 |
| HYDROGENATED JOJOBA OIL | 0.1 |
| ORYZA SATIVA (RICE) BRAN WAX | 2.8 |
| COPERNICIA CERIFERA (CARNAUBA) WAX (and) | 7 |
| PEG-30 GLYCERYL STEARATE | |
| DISTEARDIMONIUM HECTORITE | 5.8 |
| TALC | 1 |
| POLYVINYL LAURATE | 2.2 |
| ALLYL STEARATE/VA COPOLYMER | 3.3 |
| VP/EICOSENE COPOLYMER | 2 |
| ETHYLENEDIAMINE/STEARYL DIMER | 1 |
| DILINOLEATE COPOLYMER | |
| PROPYLPARABEN | 0.19 |
| METHYLPARABEN | 0.15 |
| ALCOHOL DENAT. | 2 |
| PROPYLENE CARBONATE | 1.9 |
| ISODODECANE | 47.32 |

Each of the inventive formula A and J was applied onto false eyelashes. It was visually observed that the eyelashes treated with Formula A had more volume or were thicker than the eyelashed treated with Formula J. This results means that the inventive formula had better thickening or volumizing properties than comparative formula J.

It is to be understood that the foregoing describes preferred embodiments of the invention and that modifications may be made therein without departing from the spirit or scope of the invention as set forth in the claims.

What is claimed is:

1. A cosmetic composition for making up and/or coating keratinous fibers, said composition comprising:
(a) at least one hydrocarbon film forming polymer,
(b) at least one silicone film forming polymer,
(c) at least one microcrystalline wax, present in an amount ranging from about 4% to less than or equal to 8% by weight, relative to the total weight of the composition; and
(d) at least one volatile solvent.

2. The cosmetic composition of claim 1, wherein the at least one hydrocarbon film forming polymer includes at least one hydrocarbon resin chosen from an indene hydrocarbon resin, an aliphatic pentadiene resin, a mixed pentadiene and indene resin, a polycyclopentadiene resin, a diene resin from cyclopentadiene dimers, a diene resin from isoprene dimers, a hydrogenated resin derived mainly from the polymerization of pentadiene, and mixtures thereof.

3. The cosmetic composition of claim 2, wherein the at least one hydrocarbon resin is a hydrogenated styrene/methyl styrene/indene copolymer.

4. The cosmetic composition of claim 1, wherein the at least one silicone film forming polymer includes a silicone resin chosen from a polymethylsilsesquioxane resin, a polypropylsilsesquioxane resin, a polydimethylsiloxane, a siloxysilicate resin, and mixtures thereof.

5. The cosmetic composition of claim 4, wherein the siloxysilicate resin is a trimethylsiloxysilicate resin.

6. The cosmetic composition of claim 1, wherein the at least one hydrocarbon film-forming polymer is present in the cosmetic composition in an amount ranging from about 1% to about 20% by weight, relative to the total weight of the composition.

7. The cosmetic composition of claim 1, wherein the at least one silicone film-forming polymer is present in the cosmetic composition in an amount ranging from about 0.1% to about 10% by weight, relative to the total weight of the composition.

8. The cosmetic composition of claim 1, wherein the at least one microcrystalline wax has a melting point ranging from about 63° C. to about 94° C.

9. The cosmetic composition of claim 1, wherein the at least one microcrystalline wax has a melting point ranging from about 70° C. to about 80° C.

10. The cosmetic composition of claim 1, wherein the at least one microcrystalline wax has a penetration hardness value ranging from about 4 to about 80.

11. The cosmetic composition of claim 1, wherein the at least one microcrystalline wax has a penetration hardness value ranging from about 60 to about 80.

12. The cosmetic composition of claim 1, wherein the at least one microcrystalline wax is present in the cosmetic composition in an amount ranging from about 5% to less than or equal to 7.5% by weight, relative to the total weight of the composition.

13. The cosmetic composition of claim 1, wherein the at least one microcrystalline wax is present in the cosmetic composition in an amount ranging from about 6.5% to less than or equal to 7.5% by weight, relative to the total weight of the composition.

14. The cosmetic composition of claim 1, wherein the at least one microcrystalline wax is present in the cosmetic composition in an amount of about 7.2% by weight, relative to the total weight of the composition.

15. The cosmetic composition of claim 1, wherein the at least one volatile solvent is chosen from hydrocarbon oils, silicone oils, esters and ethers.

16. The cosmetic composition of claim 1, wherein the at least one volatile solvent is isododecane.

17. The cosmetic composition of claim 1, wherein the at least one volatile solvent is present in the cosmetic composition in an amount ranging from about 10% to about 95% by weight, relative to the total weight of the composition.

18. The cosmetic composition of claim 1, further comprising at least one non-volatile oil.

19. The cosmetic composition of claim 1, further comprising at least one desired agent chosen from a colorant, a fiber, a wax other than the at least one microcrystalline wax, a cosmetically active agent, and a film forming agent other than the at least one hydrocarbon film forming polymer and the at least one silicone film forming polymer.

20. The cosmetic composition of claim 19, wherein the colorant is chosen from pigments, dyes, nacreous pigments, and pearling agents.

21. The cosmetic composition of claim 19, wherein the fiber is chosen from nylon fibers, rayon fibers, silk fibers, cotton fibers; wool fibers, flax fibers, cellulose fibers, polyamide fibers, viscose fibers, acetate fibers, acrylic fibers, polyolefin fibers, silica fibers, carbon fibers, polytetrafluoroethylene fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from mixtures of polymers, substantially rectilinear rigid fibers, elastomeric fibers, TDI/trimellitic anhydride copolymer, and mixtures thereof.

22. The cosmetic composition of claim 1, wherein the composition is a mascara composition and the keratinous fibers are the eyelashes.

23. The cosmetic composition of claim 1, wherein the composition comprises a single phase.

24. A method for making up and/or enhancing the appearance of keratinous fibers comprising applying to the keratinous fibers, the composition of claim 1.

25. A cosmetic composition for making up and/or coating keratinous fibers, said composition comprising:
(a) a hydrocarbon film-forming polymer comprising a hydrogenated styrene/methyl styrene/indene copolymer, and present in an amount ranging from about 2% to about 7.5% by weight; and
(b) a silicone film-forming polymer comprising a trimethylsiloxysilicate resin, present in an amount ranging from about 0.5% to about 3% by weight;
(c) at least one microcrystalline wax, having a melting point ranging from about 70° C. to about 80° C. and a penetration hardness value ranging from 60 to 80, and present in an amount ranging from about 6.5% to less than or equal to 7.5% by weight;
(d) a volatile solvent comprising isododecane; and
(e) at least one colorant; all weights being relative to the total weight of the composition.

26. An assembly for packaging and applying a product for coating keratinous fibers, comprising:
(1) a container comprising a cosmetic composition containing:
(a) at least one hydrocarbon film forming polymer,
(b) at least one silicone film forming polymer,
(c) at least one microcrystalline wax, present in an amount ranging from about 4% to less than or equal to 8% by weight, relative to the total weight of the composition; and
(d) at least one volatile solvent; and
(2) an applicator for applying the cosmetic composition to the fibers.
